(19) United States
(12) Patent Application Publication
(10) Pub. No.: US 2011/0112364 A1
(43) Pub. Date: May 12, 2011

(54) Minimally Invasive Surgical Apparatus in the Form of a Cannula

(76) Inventors: Rebecca J. Rone, Columbia, MO (US); Anthony N. Harris, Columbia, MO (US); Jonathan R. Thompson, Cincinnati, OH (US)

(21) Appl. No.: 12/941,436

(22) Filed: Nov. 8, 2010

Related U.S. Application Data
(60) Provisional application No. 61/280,663, filed on Nov. 6, 2009.

(51) Int. Cl. A61B 1/00

(52) U.S. Cl. 600/114

(57) ABSTRACT

A minimally invasive surgical apparatus in the form of a cannula has a length with opposite proximal and distal ends, and differing characteristics of malleability along the cannula length. A switch can be provided on the cannula adjacent the distal end. The switch can be intra-abdominally activated to selectively provide suction and/or irrigation in minimally invasive surgical procedures such as general laparoscopic surgery, single incision laparoscopic surgery, natural orifice transluminal endoscopic surgery and robotic surgery.

### CROSS-REFERENCE TO RELATED APPLICATIONS

This patent application claims the benefit of the provisional patent application No. 61/280,663, which was filed on Nov. 6, 2009.

### STATEMENT REGARDING FEDERALLY SPONSORED RESEARCH OR DEVELOPMENT

Not Applicable.

### BACKGROUND OF THE INVENTION

(1) Field of the Invention

The present invention is directed to a minimally invasive surgical apparatus in the form of a cannula that is designed for use in minimally invasive surgical procedures such as general laparoscopic surgery, single incision laparoscopic surgery, natural orifice transluminal endoscopic surgery, thoracoscopic surgery, arthroscopic surgery and robotic surgery. More specifically, the present invention is directed to a minimally invasive surgical apparatus comprising a cannula having a length with opposite proximal and distal ends and with differing characteristics of malleability and elasticity along the cannula length. A proximal portion of the cannula length is more rigid than a distal portion of the cannula length, and the distal portion of the cannula length can be moved to a particular position or into a particular shape relative to the proximal portion and substantially stay in that position or shape. The cannula is designed for use with conventional sources of suction and irrigation and their controllers. Alternatively, the cannula can be provided with an actuator at its distal end that can be activated from inside a body cavity to selectively provide suction and/or irrigation at the cannula distal end in minimally invasive surgical procedures.

(2) Description of the Related Art

In large, open incision surgery, a suction cannula connected by a length of sterile tubing to a centralized vacuum system is always available to the surgeon. If the surgeon needs to evacuate blood or other bodily fluids from a surgical site in the open incision, the surgeon or an assistant will pick up the suction cannula and direct the distal end of the cannula to the area of interest at the surgical site. Continuous suction provided by cannulas of this type has been available in operating rooms for many years. Suction cannulas used for this purpose are well known in the prior art and have been available commercially for many years.

Minimally invasive surgery is a phrase often used to describe different types of surgical procedures where the large, open incision of conventional surgery is replaced by surgical techniques that allow surgical procedures to be performed through smaller incisions. Laparoscopic surgical procedures and other minimally invasive surgical procedures typically employ a pressurized gas to create and maintain an enlarged body cavity or space for performing the surgical procedure. Pressurized gas is injected through the abdominal wall or through the layers of skin of the patient's body to insufflate and expand the abdominal wall or layers of skin and create the space or cavity inside the body for conducting the surgical procedure. Without this created space, the surgeon would not be able to freely manipulate and/or view surgical instruments and tissue in the body when conducting the surgical procedure. Other types of minimally invasive surgery that do not employ insufflation include thoracoscopic surgery and arthroscopic surgery.

Providing suction and/or irrigation in minimally invasive surgical procedures employing a distending gas is typically provided by a long, rigid cannula that is also inserted through a small incision in the abdominal wall or skin layers to position the distal end of the cannula at a desired position at the surgical site. The opposite proximal end of the cannula typically has a hand piece that has manual actuators for valves in or on the hand piece. The valves control the supply of suction and/or irrigation liquid through the cannula. Manual manipulation of the hand piece by the surgeon outside of the patient's body moves the distal end of the cannula inside the patient's body. An example of such a prior art hand piece and cannula is disclosed in the U.S. patent of Cover, et al. U.S. Pat. No. 6,652,488. This common prior art cannula provides both the functions of suction and irrigation in a single device. When suction or irrigation is desired at the surgical site, a suction or irrigation actuator on the hand piece is manually depressed and the vacuum pressure or irrigation liquid is transmitted through the cannula to the distal end of the cannula at the surgical site.

Prior art suction cannulas of the type described above have also been used in both open incision and minimally invasive surgical procedures employing electric energy surgical instruments to evacuate smoke generated by the instruments during a surgical procedure. This is particularly useful in laparoscopic surgery procedures to evacuate smoke that creates a haze that is difficult to see through to enable the surgeon to clearly view the surgical site throughout the procedure.

Extensions of laparoscopic surgery procedures that also employ suction cannula of the type described above include robotic assisted laparoscopic surgery, single incision laparoscopic surgery and natural orifice transluminal endoscopic surgery (NOTES).

The long, rigid cannulas used to provide suction and/or irrigation in minimally invasive procedures have been found to be disadvantaged in that positioning the distal end of the long, rigid cannula at a desired position at a surgical site inside a body cavity requires manual manipulation of the opposite proximal end of the long, rigid cannula from outside the body. This makes it difficult to accurately position the cannula distal end at a desired position inside the body cavity. In addition, selectively providing suction and/or irrigation to the surgical site at the distal end of the long, rigid cannula requires manual activation of actuators located on the hand piece at the opposite proximal end of the long, rigid cannula. This complicates the use of the cannula in maintaining the cannula distal end at a desired position while manipulating actuators on the cannula proximal end.

Furthermore, in traditional laparoscopic surgical procedures a 5 mm incision is made in the abdominal wall and a port is inserted in the incision. Surgical instruments used in the surgical procedure are inserted through the port. When suction is needed during the surgical procedure, the surgeon must first remove the instrument currently being used from the port, and then insert the suction cannula through the port. During general surgery, it is not uncommon for the surgeon to swap out their surgical instrument for a suction cannula an average of five to ten times during a single procedure. In order to allow the surgical instrument to remain in the port it would be necessary to make another incision in the abdomen and place an additional port in that incision.

The dissatisfaction with current suction cannula is even more apparent in robotic surgery where there is a need for greater surgeon autonomy in regard to suction control during a surgery. While the surgeon operates across the room at a counsel, an assistant must sit at the surgery table to provide retraction, suction, irrigation, insertion of sutures, entrapment and removal of dissected tissue, as well as port removal and closure.

### SUMMARY OF THE INVENTION

The present invention provides a new and improved suction and/or irrigation apparatus for minimally invasive surgery in the form of a tubular cannula that provides surgeon autonomy while reducing the size of the abdominal wall opening required for entry of the cannula through the abdomen. The apparatus is a stand-alone apparatus that provides the benefits of less operative time, does not require a separate incision and a port inserted, is surgeon controlled, can be manufactured at low cost, can be integrated with current operating room sources of suction or irrigation and does not add complexity to the laparoscopic surgical procedure or a robotic surgery system. With the elimination of a need for an additional port, the apparatus can be used without taking an instrument out of the abdomen.

The apparatus is comprised of a tubular cannula having a length with different degrees of malleability. In one embodiment of the apparatus, an actuator is provided at the distal end of the cannula length that is actuated inter-abdominally (or inside a body cavity) to selectively supply suction and/or irrigation to a minimally invasive surgical site. The apparatus of the invention allows the cannula distal end to remain inside a body cavity surgical site where the distal end can be easily manipulated by a surgical instrument to a desired location inside the body cavity and substantially stay at that location when released by the instrument. The embodiment of the apparatus having an actuator at the distal end of the cannula also allows the actuation of the actuator from inside the body cavity by a surgical instrument to selectively supply suction and/or irrigation liquid to the desired location of the surgical site.

The cannula has a length with opposite proximal and distal ends. The proximal end of the cannula is adapted to be connected with conventional suction and irrigation sources that are operated by a conventional controller for such sources where the controller includes electrically operated valves or other similar devices that are responsive to an electric signal. In some embodiments of the cannula conventional controls for the source of suction (vacuum) may be used such as mechanical valves, for example, trumpet valves. The length of the cannula has different characteristics of malleability. What is meant by a “malleable” characteristic is the ability to be shaped or formed. A more malleable portion of the cannula is easily bent or shaped where a less malleable portion resists bending or shaping. The cannula has a small exterior diameter dimension of about 3 mm in size. The small size allows the cannula to be used through a puncture site in the abdomen as opposed to an incision and port, leaving a significantly smaller scar than a trocar or port site. Other embodiments of the cannula could have an exterior diameter dimension larger than 3 mm.

In a preferred embodiment of the cannula the proximal portion or first section is substantially straight and substantially rigid. In other embodiments the proximal portion or first section of the cannula length is malleable, enabling this portion of the cannula to be inserted through the abdominal wall where the more distal portion of the cannula extending both externally and internally of the abdomen can be bent without buckling and obstructing the lumen of the cannula. In the preferred embodiment permanent deformation may occur if a shaping or bending force is applied that has enough force to bend the rigid first section of the cannula.

A distal portion or second section of the cannula length that is adjacent the distal end of the cannula has a greater characteristic or degree of malleability. In one embodiment of the cannula the distal portion or second section of the cannula can be moved by a surgical instrument to a desired location inside a body cavity and substantially stay at that location when released by the instrument.

The different characteristics of malleability in the first and second sections of the cannula can be achieved by extruding these sections of the cannula of materials having different characteristics of malleability. Alternatively, the different characteristics of malleability in the first and second sections of the cannula can be achieved by embedding a deformable wire or wires in the cannula sections where the wire or wires have different characteristics of malleability. Furthermore, instead of embedding the wire or wires in the extruded first and second sections of the cannula, the cannula could be constructed from an inner PVC tube with the wire or wires extending along the length of the exterior surface of the PVC tube, and a silicone tube positioned over the wire or wires and over the PVC tube with the silicone tube sandwiching the wire between it and the PVC tube.

In one embodiment of the cannula a tip is provided at the distal end of the cannula. The tip provides an area to be grasped by a surgical instrument manipulated by the surgeon. The tip has a gripping surface that resists the grasping instrument slipping from the tip when the tip is wet from bodily fluids.

In one embodiment of the cannula an actuator is provided in the tip of the cannula. The actuator can be any type of actuator that can control a supply of suction pressure and/or irrigation liquid in response to actuation of the actuator. For example, the actuator could be an electric switch that communicates with a controller of a source of suction or irrigation. The switch is sealed inside the cannula tip between the internal lumen of the cannula and the exterior surface of the cannula. Electrical conductors extend from the switch. The conductors are embedded in and run along the length of the cannula from the switch at the distal end of the cannula to the proximal end of the cannula. The conductors are sealed inside the cannula length between the exterior surface of the cannula and the interior surface of the cannula lumen. One or both of the electrical conductors could be the wire or wires that extend along the length of the first and second sections of the cannula and provide the different characteristics of malleability and elasticity along the first and second sections of the cannula length.

At the cannula proximal end the conductors exit the cannula and are accessible for electrical communication to a conventional controller of a suction and/or irrigation source. When the conductors are electrically connected to the controller, activation of the cannula switch transmits a signal to the controller that in turn controls an electromechanical valve of the source of suction to provide suction pressure to the cannula, or controls an electromechanical valve of the source of irrigation liquid to provide irrigation liquid to the cannula.

The switch in the cannula tip can be actuated by grasping the tip with a surgical grasper and exerting a compressive force on the tip. Alternatively, the switch can be activated by merely exerting a compressive force on one side of the tip with a separate surgical instrument. Still further, the switch could be activated by merely positioning a surgical instrument adjacent the tip. For example, the switch could be a magnetic switch that is activated by positioning a magnetic surgical instrument adjacent the tip. Additionally, a pair of separate switches could be provided at the cannula distal end, with one switch activating the source of suction and the other switch activating the source of irrigation. The two switches would be spaced along the length of the cannula distal end so that both switches could not be activated simultaneously.

In embodiments of the cannula that do not employ an electric switch, activation may be mechanically driven by a conventional trumpet valve at the cannula proximal end, by a conventional foot-operated actuator, or by a mechanical actuator provided at the cannula distal end.

### BRIEF DESCRIPTION OF THE DRAWINGS

Further features of the invention are set forth in the following detailed description of the invention and in the drawing figures.

FIG. 1 is a schematic representation of the minimally invasive surgical apparatus of the invention and an exemplary environment in which the apparatus is used.

FIG. 2 is a schematic representation of one embodiment of the apparatus of the invention.

FIG. 3 is a schematic representation of a further embodiment of the apparatus of the invention.

FIG. 4 is a schematic representation of a further embodiment of the apparatus of the invention having an actuator at its distal end.

FIG. 5 is a schematic representation of the construction of the tip of the apparatus of FIG. 4 removed from the cannula distal end, and the actuator of the apparatus inside the tip.

### DETAILED DESCRIPTION OF THE INVENTION

The minimally invasive surgical apparatus 10 of the present invention is designed for use with conventional sources of suction pressure and irrigation liquid typically employed in surgical procedures as well as their controllers. Because suction and irrigation devices of this type are known in the art, and because controllers for devices of this type are known in the art, they will not be described in detail herein.

The minimally invasive surgical apparatus 10 of the invention comprises a tubular cannula 12 having a length with opposite proximal 14 and distal 16 ends. The cannula 12 is constructed of biocompatible materials that are typically used in cannula or catheter constructions. The cannula proximal end 14 is provided with a conventional connector that will connect the cannula to standard tubing. Connecting the opposite end of the standard tubing to a source of suction pressure and/or a source of irrigation liquid communicates the inner lumen of the cannula 12 with the source of suction pressure and/or the source of irrigation liquid and thereby communicates the suction or liquid through the cannula length to the cannula distal end 16.

The length of the cannula 12 is comprised of two different sections, a first section 22 and a second section 24, in one embodiment of the cannula 12. A further embodiment of the cannula 12 includes a third section 26. The two sections 22, 24 or three sections 22, 24, 26 are continuous and the cannula has a smooth continuous exterior surface from its proximal end 14 to the distal end 16 and a smooth continuous interior surface surrounding the lumen from its proximal end 14 to its distal end 16. The sections of the cannula have distinct characteristics of malleability. As stated earlier, what is meant by the characteristic of malleability is the ability to be shaped or formed, as is the common understanding. A more malleable section of the cannula is easily bent or shaped where a less malleable section of the cannula is more difficult to bend or shape.

The cannula first section 22 extends from the cannula proximal end 14 along the cannula length toward the cannula distal end to an intermediate point 28 along the cannula length. In a preferred embodiment of the cannula 12 the first section 22 is substantially straight and substantially rigid. The first portion of the cannula length that extends along the cannula first section 22 is constructed of a biocompatible metal or polymer. In other embodiments of the cannula the first section 22 could be constructed of materials that give the cannula first section 22 a lesser characteristic of malleability and a greater characteristic of elasticity. This enables the cannula first section 22 to be positioned or inserted through an abdominal wall where the first section 22, extending both externally and internally of the abdominal wall can be bent without buckling and/or obstructing the inner lumen of the cannula. The outer diameter of the cannula first section 22 is preferably 2-3 mm and the length of the cannula first section 22 is preferable around 30 cm. The smaller external diameter dimension of the cannula 12 enables it to be inserted through the abdominal wall by making a stab puncture in the abdominal wall. This eliminates the need for a separate incision in the abdominal wall and the insertion of a trocar or port through the incision to accommodate the cannula. Preliminary studies have shown that the smaller outer diameter of the cannula 12 does not affect suction capabilities, does not injure organs, will remove blood at an efficient rate and is efficient at breaking up a clot. In other embodiments of the cannula 12 the outer diameter dimension could be larger than 3 mm.

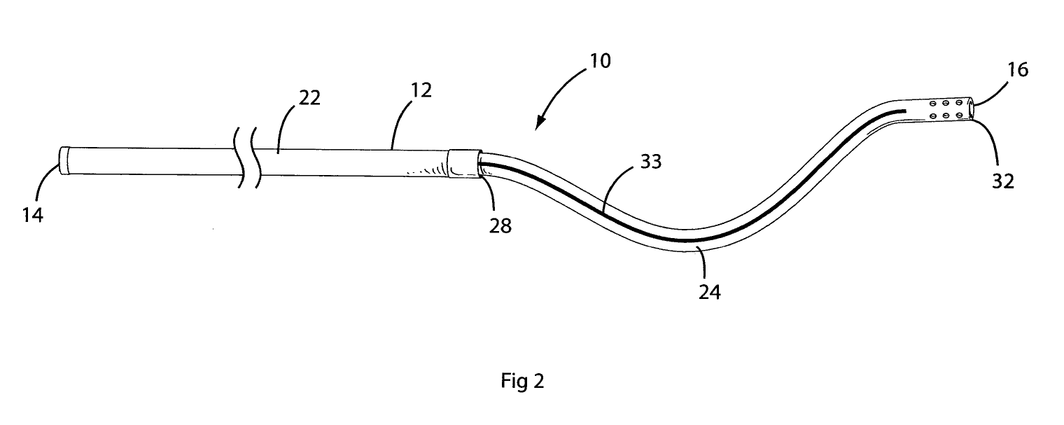

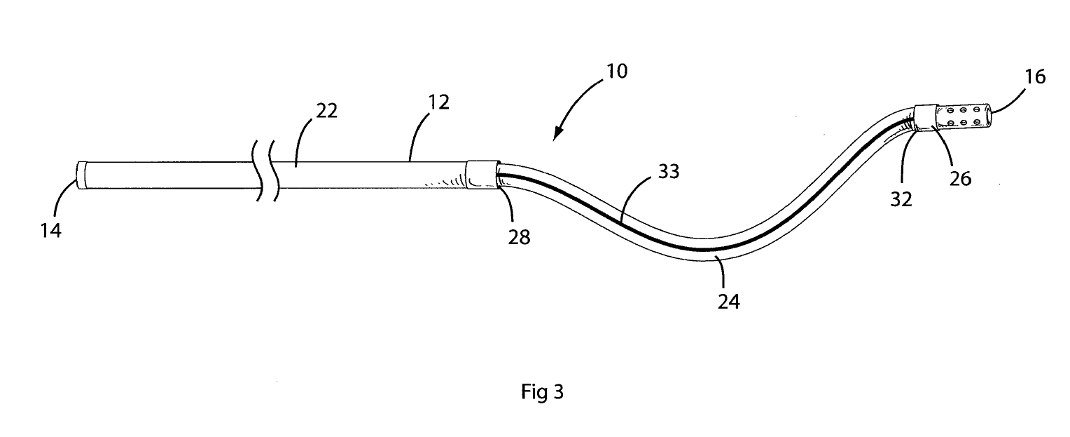

The cannula second section 24 extends from the cannula first section 22 or from the intermediate point 28 on the cannula length toward the cannula distal end 16 to a distal end 32 of the second section 24. In the embodiment of the cannula 12 shown in FIG. 2 the distal end 32 of the second section 24 is also the distal end 16 of the cannula 12. In the embodiment of the cannula 12 shown in FIG. 3 the distal end 32 of the second section 24 is an additional intermediate point 32 on the length of the cannula. The cannula second section 24 has an outer diameter dimension of 2-3 mm and a length of around 10 cm. This portion of the cannula length occupied by the cannula second section 24 has a greater characteristic of malleability than the cannula first section 22. Preferably, the cannula second section 24 is substantially inelastic. This enables the cannula second section 24 to function as a joint between the cannula proximal end 14 and the cannula distal end 16. The greater malleability of the cannula second section 24 limits or substantially reduces any forces transmitted from the cannula distal end 16 to the cannula first section 22 that are the result of movement of the cannula distal end 16. The inelastic characteristic of the cannula second section 24 also enables the cannula distal end 16 to be freely manipulated with a surgical instrument within the body cavity operative space without experiencing any restrictions to the movement of the cannula distal end 16 from the less malleable or rigid characteristics of the cannula first section 22. The cannula second section 24 can be formed as a continuous extrusion of materials having a greater characteristic of malleability than the cannula first section 22. Alternatively, the cannula second section 24 can be formed as a continuous extension from the cannula first section 22 and of a biocompatible polymer that is substantially inelastic with a length of wire 33 extruded in the cannula second section 24. The gauge of the wire 33 would be less than the wall thickness of the cannula second section 24. Alternatively, a lumen for the wire 33 could be left in the cannula second section 24 and the wire 33 snaked through the lumen. The wire 33 extending through the cannula second section 24 would enable the cannula second section to be moved to a desired position or shape inside the body cavity by the surgeon grasping the cannula with an instrument adjacent the second section distal end 32, with the wire 33 holding the cannula second section substantially in the position or shape after the instrument is removed from the second section. Still further, rather than embedding a deformable wire 33 in the extruded length of the cannula second section 24, the cannula second section 24 could be comprised of an inner tube covered with an outer tube with the deformable wire 33 sandwiched in between. For example, the cannula second section 24 could be constructed of an inner PVC tube with the deformable wire 33 laying along the exterior surface of the length of the PVC tube, and with a silicone tube surrounding the deformable wire 33 and the PVC tube and extending along the length of both the wire 33 and the PVC tube. Still further, the cannula second section 24 could be formed from an inner tube and an outer tube as described above, with a pair of deformable wires 33 extending between the two tubes and along the length of the cannula second section 24. The wire 33 or pair of wires 33 provide the ability to the cannula second section 24 to remain inside a body cavity and be moved to a desired position or into a particular shape relative to the body cavity and the cannula first section 22 by a surgical instrument held by the surgeon, and substantially remain in the position or shape when released by the instrument. The wire 33 or pair of wires 33 also may function as one or a pair of electrical conductors for the actuator at the cannula distal end.

The embodiment of the cannula 12 shown in FIG. 3 has the same construction as the embodiment of FIG. 2 described above with the addition of the third section 26. The third section 26 of the cannula length is a tubular tip. The portion of the cannula length defined by the third section 26 is less malleable than the second section 24 of the length of the cannula. The cannula third section 26 extends from the cannula second section 24 or the additional intermediate point 32 on the cannula length to the cannula distal end 16. The cannula third section 26 has an outer diameter of 2-3 mm and a length of about 3 cm. The tip of the cannula third section 26 provides an exterior gripping surface that can be grasped by a surgical instrument and resist slipping of the instrument even when the surface is wet.

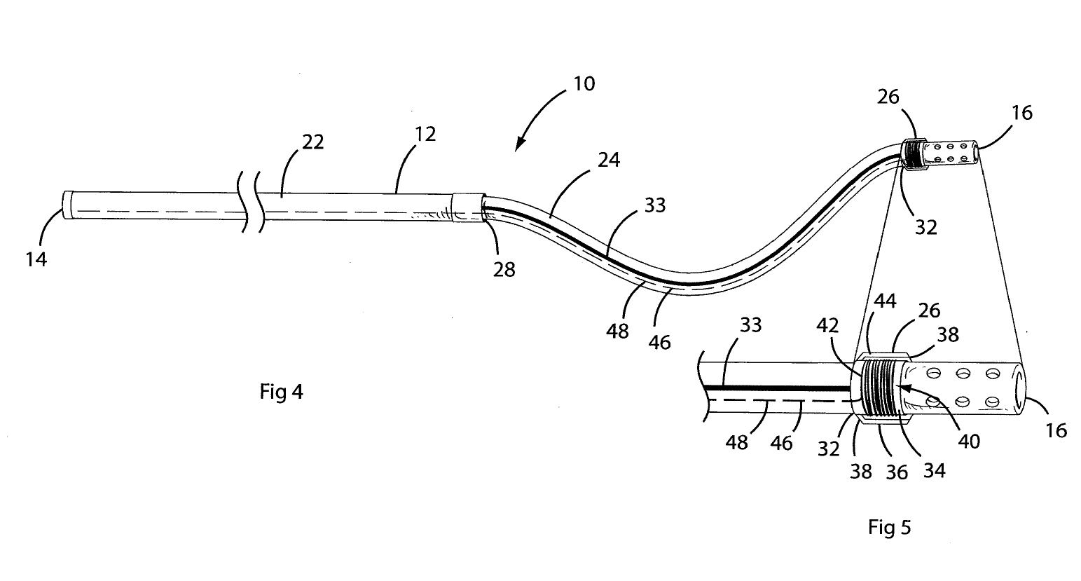

FIG. 4 is a representation of an embodiment of the cannula 12 having an actuator on the third section. FIG. 5 is an enlarged, partial view of the cannula 12 of FIG. 4. In FIG. 5, the cannula third section 26 is shown comprised of an inner tubular member 34 and an outer tubular member 36 that are connected together at their opposite ends by annular seals 38. This construction creates a cylindrical interior void or volume within the cannula third section 26 between the inner 34 and outer 36 tubular members. The inner cylindrical volume of the cannula third section 26 is sealed by the annular seals 38 at the opposite ends of the third section. The inner tubular member 34 is more rigid than the outer tubular member 36. The outer tubular member 36 is malleable and elastic and can be deflected radially inwardly toward the inner tubular member by a moderate force exerted on the exterior surface of the cannula third section. Alternatively, the third section 26 of the cannula could be comprised of an inner tube 34 that is an extension of the inner tube of the cannula second section 24 described above, and an outer tube 36 that is an extension of the outer tube of the cannula second section 24 discussed above. The two deformable wires 33 that extend through this embodiment of the cannula second section 24 described above would then also be employed as the electrical connectors to the actuator.

The embodiment of minimally invasive surgical apparatus of the invention shown in FIG. 5 also comprises an actuator 40 that is sealed inside the cylindrical interior volume of the cannula third section 26. The actuator can be any type of actuator that can control a supply of suction pressure and/or irrigation liquid in response to actuation of the actuator. For example, the actuator could be an electric switch that communicates with a controller of a source of suction or irrigation. The actuator 40 could also be a pneumatic actuator, or a pressure switch type actuator, or a mechanical actuator. An exemplary embodiment of the actuator as a switch 40 is schematically shown in FIG. 5. The switch 40 comprises a plurality of spaced electrically conductive rings 42 arranged along the length of the cannula third section 26, and a plurality of parallel bars 44 that extend along the length of the cannula third section 26. The conductive rings 42 are supported on an outer surface of the inner tubular member 34 of the cannula third section 26 and the conductive bars 44 are supported on an inner surface of the outer tubular member 36 of the cannula third section 26. The rings 42 and bars 44 are spaced radially from each other on opposite sides of the cylindrical interior bore of the cannula third section 26. In other embodiments of the apparatus the actuator could be located at different positions along the cannula length and is not limited to being positioned at the distal end of the cannula length.

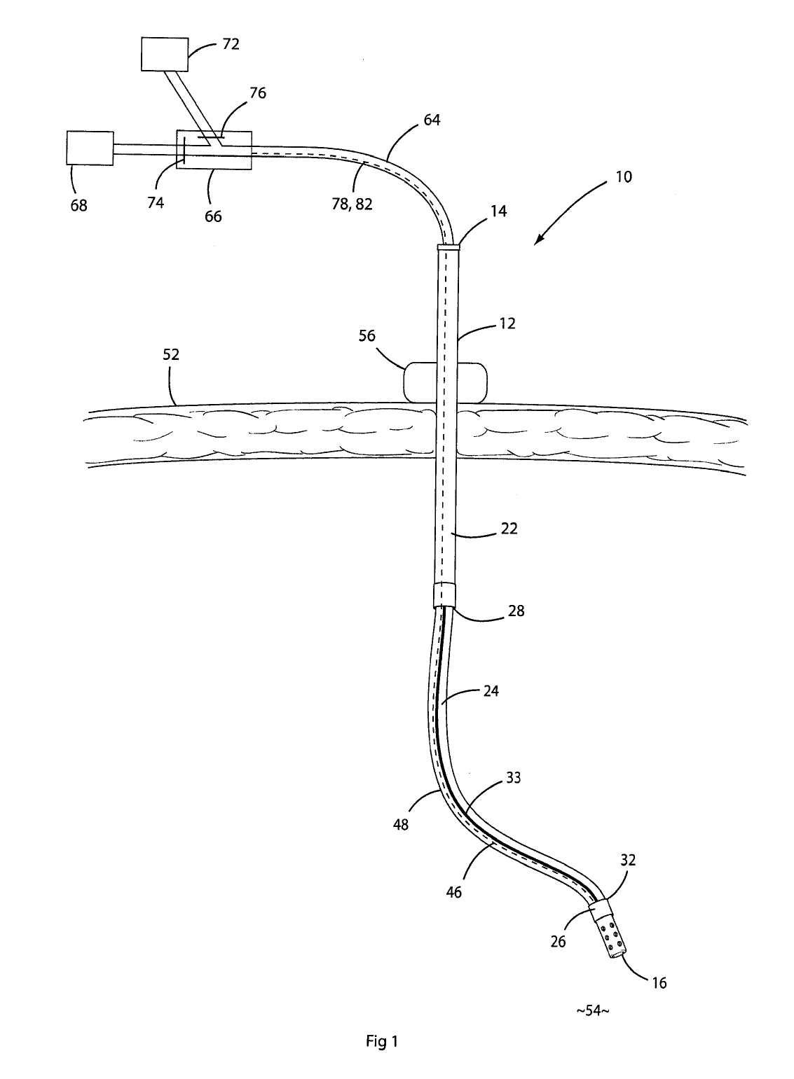

An electrical conductor 46 is connected to each of the electrically conductive rings 42 and a separate electrical conductor 48 is connected to each of the electrically conductive bars 44. The electrical conductors 46, 48 are represented schematically by the dashed line shown in FIGS. 1, 4 and 5. These two electrical conductors 46, 48 extend from the cannula third section 26, through the cannula second section 24, through the cannula first section 22 and exit the cannula 12 at the cannula proximal end 14. Preferably, the electrical conductors 46, 48 are embedded in the cannula 12 between the interior surface of the lumen and the exterior surface of the cannula 12. This construction seals the electrical conductors 46,48 along the entire length of the cannula.

Exerting a moderate force on the exterior of the cannula third section 26 will move at least one of the electrically conductive bars 44 inside the cannula third section 26 radially inwardly until it makes contact with at least one of the electrical conductive rings 42. This contact between the bar 44 and ring 42 completes a circuit through the switch in the cannula third section. The electrical conductors 46,48 transmit an electric signal through the length of the cannula 12 in response to actuation of the switch. Actuating the switch 40 can be accomplished by exerting a compressive force on the cannula third section 26 by grasping the cannula third section 26 between the jaws of a surgical grasper. Alternatively, this could be accomplished by exerting a force on the exterior of the cannula third section 26 that is sufficient to move one of the conductive bars 44 into contact with one of the conductive rings 42. Still further, a variant of the exemplary switch depicted in FIG. 5 could be employed in the cannula third section 26 that is responsive to merely positioning a surgical instrument adjacent to the cannula third section 26, for example a magnetic switch that is responsive to a magnetic surgical instrument positioned in close proximity to the switch.

FIG. 1 shows one exemplary environment of the apparatus of the invention 10 being employed in a laparoscopic surgical procedure. As depicted in FIG. 1, the cannula 12 has been inserted through the abdominal wall 52 and the cannula first section 22 is positioned traversing the abdominal wall 52. The cannula third section 26 has been positioned adjacent an intra-abdominal surgical site 54. The greater degree or characteristic of malleability of the cannula second section 24 allows the cannula third section 26 to remain at its placed position.

In the exemplary environment shown in FIG. 1, the cannula first section 22 is shown held in place traversing the abdominal wall by a restrictive or stabilizing element 56. The stabilizing element 56 is constructed as a thick, disc-shaped structure of a foam or other similar material having moderate elasticity. The cannula first section 22 is inserted through a center aperture of the disc-shaped stabilizing element 56 and is held there solely by friction engagement. The stabilizer element 56 may be secured and sealed to the exterior of the abdominal wall by adhesives, sutures, surgical staples or other equivalent means. The friction engagement between the cannula first section 22 and the stabilizer element 56 enables the cannula 12 to be moved in and out of the abdomen through the stabilizing element 56 and rotated relative to the abdomen while the stabilizing element limits the pitch and yaw of the cannula first section 22. As an alternative to the stabilizing element 56, a ball and socket construction may be used.

Due to the small outer diameter dimensions of the cannula 12, a stabilizing element such as those discussed above may not be necessary. It is contemplated that the cannula 12 will be inserted through the abdominal wall with a stab puncture much like that created by a suture passer. A sharp stylette will be placed through the lumen of the cannula 22 so that the stylette tip projects from the cannula distal end 16. The stylette tip will be used to puncture through the abdominal wall followed by the cannula 12 passing through the puncture. From outside the abdomen, the surgeon will then remove the stylette from the lumen of the cannula 12. It is also contemplated that with a stab incision or puncture, the elasticity of the skin around the cannula 12 will provide sufficient constriction that a separate stabilizing element will not be needed for stability. In other embodiments the cannula could have an outer diameter dimension larger than 3 mm.

A length of conventional, flexible suction tubing 64 is connected to the cannula proximal end 14. The opposite end of the tubing 64 is connected to a conventional controller 66 that communicates with a source of vacuum pressure 68. The controller 66 selectively controls the supply of vacuum pressure from the vacuum pressure source 68 to the length of tubing 64. The controller 66 also communicates with a source of irrigation liquid 72 and controls the supply of irrigation liquid to the length of tubing 64. The controller 66 is conventional and can be any type of controller known in the art. In the exemplary environment shown in FIG. 1, the controller 66 includes a pair of electrically operated valves 74, 76 that respectively control the supply of suction pressure and irrigation liquid to the tubing 64.

A pair of electrical conductors 78, 82 are represented by a dashed line extending from the cannula proximal end 14 to the controller 66. The electrical conductors 78, 82 are connected with the electrical conductors 46, 48 of the cannula 12 and provide electrical communication between the cannula switch 40 and the controller valves 74, 76. In the illustrative example of FIG. 1, depending on whether the source of suction pressure or the source of irrigation liquid is activated by the controller 66, actuation of the cannula switch 40 transmits a signal through the cannula electrical conductors 46, 48 and through the pair of electrical conductors 78, 82 to the controller 66 that results in the opening of one of the controller valves 74, 76 that in turn controls the supply of suction pressure or irrigation liquid through the flexible tubing 64 and the length of the cannula 12 to the cannula distal end 16.

With the construction of the minimally invasive surgical apparatus described above, a cannula is provided with a switch at its distal end that is actuated at a laparoscopic surgery site to selectively supply suction or irrigation liquid to the surgery site from a suction source or an irrigation liquid source external to the surgery site. In addition, the cannula of the invention described above allows the cannula distal end to remain at a desired position relative to the laparoscopic surgery site.

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. A minimally invasive surgical apparatus comprising:
a tubular cannula having a length with opposite proximal and distal ends, the cannula length being comprised of two distinct sections including a first section that extends along a first portion of the cannula length from the cannula proximal end to an intermediate point along the cannula length and a second section that extends along a second portion of the cannula length from the intermediate point on the cannula length toward the cannula distal end, the cannula first section being malleable along the first portion of the cannula length and the cannula second section being more malleable than the cannula first section along the second portion of the cannula length, whereby the cannula second section can be moved to a desired position relative to the cannula first section in response to an external force being applied to the cannula second section and the cannula second section will substantially remain in the desired position relative to the cannula first section when the external force is removed from being applied to the cannula second section.

2. The apparatus of claim 1, further comprising:
the malleability of the cannula second section enabling the second section to be moved to a particular shape relative to the cannula first section in response to the external force being applied to the cannula second section and substantially remain in the particular shape relative to the cannula first section when the external force is removed.

3. The apparatus of claim 1, further comprising:
a nonslip, exterior surface on the cannula length adjacent the cannula distal end that enables a nonslip engagement of the nonslip exterior surface by a surgical instrument to move the cannula second section in response to movement of the surgical instrument.

4. The apparatus of claim 1, further comprising:
the cannula first section being substantially rigid.

5. The apparatus of claim 4, further comprising:
the cannula first section being substantially straight.

6. The apparatus of claim 1, further comprising:
a malleable wire on the cannula extending along the cannula second section, the malleable wire being bendable in response to the cannula second section being moved to the desired position and the malleable wire holding the cannula second section substantially in the desired position.

7. The apparatus of claim 1, further comprising:
the cannula length being comprised of three distinct sections including a third section that extends along a third portion of the cannula length from the cannula second section to the cannula distal end.

8. A minimally invasive surgical apparatus comprising:
a tubular cannula having a flexible length with opposite proximal and distal ends, the cannula length being comprised of three distinct sections including a first section that extends along a first portion of the cannula length from the cannula proximal end, a second section that extends along a second portion of the cannula length from the first section toward the cannula distal end, and a third section that extends along a third portion of the cannula length from the second section to the cannula distal end, the cannula first section being malleable along the first portion of the cannula length, the cannula second section being more malleable than the cannula first section along the second portion of the cannula length, and the cannula third section being less malleable than the cannula second section along the third portion of the cannula length, whereby the cannula second section can be moved to a desired position relative to the cannula first section in response to an external force being applied to the cannula third section and the cannula second section will substantially remain in the desired position relative to the cannula first section when the external force is removed from being applied to the cannula third section.

9. The apparatus of claim 8, further comprising:
the cannula length consisting essentially of the first, second and third sections of the cannula.

10. The apparatus of claim 8, further comprising:
a nonslip exterior surface on the cannula third section that enables a surgical instrument to engage in a nonslip engagement with the nonslip exterior surface and move the cannula second section to the desired position.

11. The apparatus of claim 8, further comprising:
the cannula first section being substantially rigid.

12. The apparatus of claim 11, further comprising:
the cannula first section being substantially straight.

13. The apparatus of claim 8, further comprising:
a malleable wire on the cannula extending along the cannula second section, the malleable wire being bendable in response to the cannula second section being moved to the desired position and the malleable wire holding the cannula second section substantially in the desired position.

14. The apparatus of claim 8, further comprising:
an actuator on the cannula third section;
the cannula proximal end being adapted for communication with a source of suction pressure that is separate from the cannula to conduct suction pressure through the cannula length to the cannula distal end; and,
the actuator being connectable in an electric circuit of a controller that controls the source of suction pressure.

15. The apparatus of claim 8, further comprising:
an actuator on the cannula third section;
the cannula proximal end being adapted for communication with a source of irrigation liquid that is separate from the cannula to conduct irrigation liquid through the cannula length to the cannula distal end; and
the actuator being connectable in an electric circuit of a controller that controls the source of irrigation liquid.

16. The apparatus of claim 8, further comprising:
the actuator being contained inside the cannula third section.

17. The apparatus of claim 8, further comprising:
electrical conductors electrically communicating with the actuator and extending along the cannula length from the actuator toward the cannula proximal end.

18. A minimally invasive surgical apparatus comprising:
a tubular cannula having a length with opposite proximal and distal ends;
an electric switch on the cannula adjacent the cannula distal end; and
a pair of electrical conductors each having a length with opposite proximal and distal ends, the electrical conductor distal ends being electrically connected to the switch and the electrical conductor lengths extending from the switch and along the length of the cannula to the cannula proximal end.

19. The apparatus of claim 18, further comprising:
the cannula proximal end being adapted for communication with a source of suction pressure that is separate from the cannula to conduct suction pressure through the cannula length to the cannula distal end; and
the proximal ends of the pair of electrical conductors being adapted for electric communication with a controller that controls the source of suction pressure.

20. The apparatus of claim 18, further comprising:
the cannula proximal end being adapted for communication with a source of irrigation liquid that is separate from the cannula to conduct irrigation liquid through the cannula length to the cannula distal end; and
the proximal ends of the pair of electrical conductors being adapted for electric communication with a controller that controls the source of irrigation liquid.

21. The apparatus of claim 18, further comprising:
the electric switch being contained inside the cannula at the cannula distal end.

22. The apparatus of claim 21, further comprising:
the electric conductor distal ends being contained inside the cannula and the electrical conductor lengths being contained inside the cannula.

23. The apparatus of claim 18, further comprising:
the cannula having different characteristics of malleability along the cannula length.

24. A minimally invasive surgical apparatus comprising:
a tubular cannula having a length with opposite proximal and distal ends; and
a switch on the cannula, the switch being connectable to an electric circuit where at least a portion of the electric circuit is separate from the apparatus, the switch being movable to a closed condition to close the electric circuit when the switch is connected in the electric circuit and the switch being movable to an opened condition to open the electric circuit when the switch is connected in the electric circuit, the switch being movable to the closed condition in response to an object separate from the cannula contacting the cannula.

25. The apparatus of claim 24, further comprising:
the switch being movable to the closed condition in response to the object separate from the cannula exerting a compressive force on an exterior surface of the cannula.

26. The apparatus of claim 24, further comprising:
the switch being adjacent the cannula distal end and the switch being movable to the closed condition in response to the object separate from the cannula contacting the cannula adjacent the cannula distal end.

27. The apparatus of claim 26, further comprising:
the switch being movable to the closed condition in response to the object separate from the cannula exerting a compressive force on an exterior surface of the cannula.

28. The apparatus of claim 24, further comprising:
the switch being movable from the closed condition to the opened condition in response to the object separate from the cannula being removed from contacting the cannula.

29. The apparatus of claim 24, further comprising:
the cannula proximal end being adapted for communication with a source of suction pressure that is separate from the cannula to conduct suction pressure through the cannula length to the cannula distal end; and
the switch being connectable in an electric circuit of a controller that controls the source of suction pressure.

30. The apparatus of claim 24, further comprising:
the cannula proximal end being adapted for communication with a source of irrigation liquid that is separate from the cannula to conduct irrigation liquid through the cannula length to the cannula distal end; and
the switch being connectable in an electric circuit of a controller that controls the source of irrigation liquid.

31. The apparatus of claim 24, further comprising:
the electric switch being contained inside the cannula at the cannula distal end.

32. The apparatus of claim 24, further comprising:
the cannula having different characteristics of malleability along the cannula length.
